(19) United States
(12) Patent Application Publication
(10) Pub. No.: US 2022/0008507 A1
(43) Pub. Date: Jan. 13, 2022

(54) METHODS AND COMPOSITIONS FOR TREATMENT OF CORONAVIRUS INFECTION AND ASSOCIATED COAGULOPATHY

(71) Applicant: UNIVERSITÄTSMEDIZIN DER JOHANNES GUTENBERG-UNIVERSITÄT MAINZ, Mainz (DE)

(72) Inventors: Wolfram RUF, Mainz (DE); Karl LACKNER, Mainz (DE); Nadine MÜLLER-CALLEJA, Lörzweiler (DE)

(21) Appl. No.: 17/487,099

(22) Filed: Sep. 28, 2021

Related U.S. Application Data
(63) Continuation of application No. PCT/IB2021/054549, filed on May 25, 2021.
(60) Provisional application No. 63/030,217, filed on May 26, 2020.
(60) Provisional application No. 63/167,535, filed on Mar. 29, 2021.

(51) Int. Cl. A61K 38/17; A61P 7/02; A61P 31/14

(52) U.S. Cl. CPC A61K 38/1767; A61P 31/14; A61P 7/02

(57) ABSTRACT

The current disclosure provides methods and compositions for treatment of SARS-CoV-2 infection and associated conditions, including COVID-19 Associated Coagulopathy. Certain aspects of the disclosure are directed to methods for treatment of SARS-CoV-2 infection comprising administering a composition comprising a therapeutically effective amount of NAPc2. Further aspects include pharmaceutical compositions comprising NAPc2 and, in some cases, one or more additional anticoagulants.

### CROSS-REFERENCE TO RELATED APPLICATIONS

This application claims the benefit of priority of U.S. Provisional Patent Application No. 63/167,535, filed Mar. 29, 2021, and U.S. Provisional Patent Application No. 63/030,217, filed May 26, 2020, which applications are incorporated by reference herein in their entirety.

### SEQUENCE LISTING

The instant application contains a Sequence Listing which has been submitted in ASCII format and is hereby incorporated by reference in its entirety. Said ASCII copy, created on May 21, 2021, is named ARCA_P0061WO_Sequence Listing.txt and is 41,035 bytes in size.

### BACKGROUND

### I. Field of the Invention

Aspects of this invention relate to at least the fields of virology, immunology, hematology, and medicine.

### II. Background

As the coronavirus disease (COVID-19) epidemic has progressed, serious complications relating to immune and inflammatory response have been increasingly observed in patients with COVID-19 illness, such as thrombosis, including stroke, and biomarker evidence of a severe coagulopathy associated with poor outcome. As is now apparent from several studies, COVID-19 illness leads preferentially to a prolongation of the prothrombin time (PT). Evidence of a severe coagulopathy and thrombotic complications such as pulmonary embolism and stroke have become hallmarks of severe COVID-19 infections. The most reliable coagulation biomarker is the D-Dimer test, which reaches very high levels in many COVID-19 patients (e.g., 16,000-20,000 μg/L), indicating they are undergoing a coagulopathy. This 103111444.1 syndrome is so frequently observed in COVID-19 that it has received the name of COVID-19 Associated Coagulopathy (CAC).

There exists a need for methods and compositions for treatment of subjects with a SARS-CoV-2 infection, including those suffering from associated symptoms and conditions such as CAC.

### SUMMARY

The current disclosure fulfils certain needs by providing methods and compositions for treating or preventing a SARS-CoV-2 infection and/or associated conditions. Accordingly, aspects of the disclosure provide methods and compositions for treating a subject for a SARS-CoV-2 infection and/or COVID-19 Associated Coagulopathy.

Embodiments of the present disclosure include methods for treating a subject having a SARS-CoV-2 infection, methods for treating a subject for COVID-19 associated coagulopathy (CAC), methods for diagnosis, methods for evaluating an efficacy of a SARS-CoV-2 treatment, pharmaceutical compositions, polypeptides, polynucleotides, and nucleic acids. Methods of the disclosure can include at least 1, 2, 3, or more of the following steps: diagnosing a subject for a SARS-CoV-2 infection, measuring one or more symptoms of a SARS-CoV-2 infection in a subject, detecting antiphospholipid antibodies in a biological sample from a subject, detecting anti-cardiolipin antibodies in a biological sample from a subject, measuring a D dimer level in a subject, diagnosing a subject with a coagulopathy, measuring a fibrinogen level in a subject, measuring an interleukin-6 level in a subject, diagnosing a subject for thrombosis, diagnosing a subject for disseminating intravascular coagulation, providing NAPc2 to a subject, providing a NAPc2 variant to a subject, providing rNAPc2 to a subject, providing an anticoagulant to a subject, and providing a coagulation factor to a subject. It is specifically contemplated that one or more of the preceding steps may be omitted in certain embodiments.

Disclosed herein, in some embodiments, is a method for treating a subject for a SARS-CoV-2 infection, the method comprising providing to the subject a therapeutically effective amount of a pharmaceutical composition comprising nematode anticoagulant protein c2 (NAPc2) or NAPc2/proline. Additional embodiments of the disclosure are directed to a method for treating a subject for COVID-19 associated coagulopathy (CAC), the method comprising providing to the subject a therapeutically effective amount of a pharmaceutical composition comprising nematode anticoagulant protein c2 (NAPc2) or NAPc2/proline.

In some embodiments, the pharmaceutical composition comprises NAPc2. In some embodiments, the pharmaceutical composition comprises recombinant NAPc2 (rNAPc2). In some embodiments, the pharmaceutical composition comprises NAPc2/proline. The pharmaceutical composition may comprise one or more additional therapeutics. In some embodiments, the method further comprises providing an additional antiviral therapy to the subject. In some embodiments, the additional antiviral therapy is remdesivir, COVID-19 convalescent plasma, an anti-SARS-CoV-2 spike protein antibody, or any combination thereof. In some embodiments, the pharmaceutical composition does not comprise any additional therapeutics. The pharmaceutical composition may comprise one or more pharmaceutically acceptable excipients.

In some embodiments, the subject was diagnosed with a SARS-CoV-2 infection. The subject may be diagnosed with a SARS-CoV-2 infection by any means known in the art including, for example, reverse transcriptase polymerase chain reaction (RT-PCR). In some embodiments, the subject was determined to have one or more symptoms of a SARS-CoV-2 infection. A symptom of a SARS-CoV-2 infection may be, for example, fever, dry cough, fatigue, loss of appetite, sore throat, diarrhea, loss of taste, or loss of smell. In some embodiments, the pharmaceutical composition is provided to the subject following the onset of the symptoms. In some embodiments, the subject was not diagnosed with a SARS-CoV-2 infection. In some embodiments, the pharmaceutical composition is provided prior to the onset of any symptoms of a SARS-CoV-2 infection. For example, the pharmaceutical composition may be provided to subject at risk for having or developing a SARS-CoV-2 infection. In some embodiments, the subject was determined to have antiphospholipid antibodies. In some embodiments, the method further comprises detecting the presence of antiphospholipid antibodies in the subject.

In some embodiments, the subject was previously treated for a SARS-CoV-2 infection. In some embodiments, the subject was determined to be resistant to the previous treatment. In some embodiments, the subject was not previously treated for a SARS-CoV-2 infection. In some embodiments, the subject is treated with a pharmaceutical composition comprising NAPc2 or NAPc2/proline together with 1, 2, 3, 4, 5, 6, 7, or more additional therapeutics (e.g., antivirals, anticoagulants, etc.).

In some embodiments, the pharmaceutical composition is provided via subcutaneous injection. In some embodiments, the pharmaceutical composition is provided via intravenous infusion. In some embodiments, the pharmaceutical composition is provided to the subject every 1, 2, 3, 4, 5, 6, 7, 8, 9, 10, 12, 13, or 14 days. In some embodiments, the pharmaceutical composition is provided to the subject every other day. In some embodiments, the pharmaceutical composition is provided on a first, second, third, fourth, fifth, sixth, seventh, eighth, ninth, tenth, eleventh, twelfth, thirteenth, and/or fourteenth day. In some embodiments, the pharmaceutical composition is provided on a first day, a third day, and a fifth day.

In some embodiments, the NAPc2 or NAPc2/proline is provided to the subject at a dose of at least, at most, or about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, or 15.0 μg/kg. In some embodiments, the NAPc2 or NAPc2/proline is provided at a dose of between 5 μg/kg and 10 μg/kg. In some embodiments, the NAPc2 or NAPc2/proline is provided at a dose of about 10 μg/kg. In some embodiments, the NAPc2 or NAPc2/proline is provided at a dose of about 7.5 μg/kg. In some embodiments, the NAPc2 or NAPc2/proline is provided at a dose of about 5 μg/kg. In some embodiments, the NAPc2 or NAPc2/proline is provided on a first day, a third day, and a fifth day. In some embodiments, the NAPc2 or NAPc2/proline is provided at a dose of about 7.5 μg/kg on a first day, 5 μg/kg on a third day, and 5 μg/kg on a fifth day.

In some embodiments, the subject is suffering from a coagulopathy. In some embodiments, the coagulopathy is COVID-19 associated coagulopathy (CAC). In some embodiments, the subject was diagnosed for a coagulopathy using one or more diagnostic tests such as, for example, a D dimer test, a fibrinogen test, a peripheral blood count, a prothrombin time (PT) test, an activated partial thromboplastin time (aPTT) test, and a thrombin time (TT) test. In some embodiments, the subject was determined to have an elevated D dimer level relative to a control or healthy subject. An elevated D dimer level may be, for example, at least 10000, 11000, 12000, 13000, 14000, 15000, 16000, 17000, 18000, 19000, or 20000 μg/L. In some embodiments, the subject was determined to have an elevated fibrinogen level relative to a control or healthy subject. In some embodiments, the subject is not suffering from a coagulopathy.

In some embodiments, the subject is suffering from disseminating intravascular coagulation (DIC). In some embodiments, the subject was diagnosed for DIC using one or more diagnostic tests such as, for example, a D dimer test, a fibrinogen test, a peripheral blood count, a PT test, and an aPTT test. In some embodiments, the subject is not suffering from DIC. In some embodiments, the subject is suffering from thrombosis. In some embodiments, the subject was diagnosed for thrombosis using one or more diagnostic test. In some embodiments, the subject is not suffering from thrombosis.

In some embodiments, the subject was previously treated for a coagulopathy. In some embodiments, the subject was previously treated with an anticoagulant. In some embodiments, the anticoagulant is a vitamin K epoxide reductase complex 1 (VKORC1) inhibitor, a thrombin inhibitor, or a factor Xa inhibitor. In some embodiments, the anticoagulant is warfarin, heparin, rivaroxaban, dabigatran, apixaban, or edoxaban. The subject may have been previously treated with 1, 2, 3, 4, 5, or more anticoagulants. In some embodiments, the subject was determined to be resistant to the previous treatment.

In some embodiments, the method further comprises providing an additional anticoagulant to the subject. In some embodiments, the additional anticoagulant is a VKORC1 inhibitor, a thrombin inhibitor, or a factor Xa inhibitor. In some embodiments, the additional anticoagulant is warfarin, heparin, rivaroxaban, dabigatran, apixaban, or edoxaban. The method may comprise providing 1, 2, 3, 4, 5, or more additional anticoagulants.

In some embodiments, the method further comprises providing a coagulation factor to the subject. In some embodiments, a coagulation factor is provided to the subject prior to, during, and/or after performing a surgery on the subject. In some embodiments, the coagulation factor is recombinant factor VIIa.

Also disclosed herein, in some embodiments, is a method for treating a subject for a SARS-CoV-2 infection, the method comprising (a) detecting the presence of antiphospholipid antibodies in a biological sample from the subject; and (b) administering a therapeutically effective amount of an antiviral therapy to the subject. In some embodiments, the antiviral therapy is NAPc2 or NAPc2/proline. In some embodiments, the antiviral therapy is remdesivir, COVID-19 convalescent plasma, an anti-SARS-CoV-2 spike protein antibody, or a combination thereof. In some embodiments, the antiphospholipid antibodies comprise anticardiolipin IgG. In some embodiments, detecting the antiphospholipid antibodies comprises an enzyme linked immunosorbent assay (ELISA). Further disclosed are methods for identifying a subject as having a SARS-CoV-2 infection comprising detecting the presence of antiphospholipid antibodies in a biological sample from the subject.

It is contemplated herein that the disclosed methods and compositions may be used for treatment of a subject for a viral infection. In some embodiments, disclosed herein is a method for treating a subject for a viral infection comprising providing to the subject a therapeutically effective amount of a pharmaceutical composition comprising NAPc2 or NAPc2/proline. A viral infection may be infection with a DNA virus. A viral infection may be infection with an RNA virus. In some embodiments, the RNA virus is a coronavirus. In some embodiments, the RNA virus is not a coronavirus. Disclosed herein, in some embodiments, is a method for treating a subject for a coronavirus infection, the method comprising providing to the subject a therapeutically effective amount of a pharmaceutical composition comprising NAPc2 or NAPc2/proline. In some embodiments, the coronavirus is a Betacoronavirus. In some embodiments, the coronavirus is a Sarbecovirus. In some embodiments, the coronavirus is a severe acute respiratory syndrome-related coronavirus. In some embodiments, the coronavirus is severe acute respiratory syndrome coronavirus (SARS-CoV) or severe acute respiratory syndrome coronavirus 2 (SARS-CoV-2). In some embodiments, the coronavirus is SARS-CoV-2.

Because the SARS-CoV-2 virus causes COVID-19, any embodiment discussed in the context of SARS-CoV-2 can be implemented with respect to COVID-19.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. It is contemplated that embodiments described herein in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B show induction of tissue factor (TF) and TNF-α (TNF) production (FIG. 1A) and ROS production measured by 2′,7′-dichlorodihydrofluorescein diacetate (H₂DCFDA) fluorescence (FIG. 1B) in human monocytic MM1.

FIGS. 2A and 2B show NAPc2 inhibition of tissue factor (TF) and TNF-α (TNF) production by antiphospholipid antibodies (aPL) in human monocytes after 3 hours (FIG. 2A) and 1 hour (FIG. 2B) of aPL treatment.

FIG. 3 shows inhibition of monocyte ROS production by NAPc2 measured by H₂DCFDA fluorescence.

FIGS. 4A-4C show aPL titers of COVID-19 patient sera. FIG. 4A shows anti-cardiolipin IgG measured using an in house ELISA. FIG. 4B shows anti-cardiolipin IgG measured using a BIO-FLASH® assay. FIG. 4C shows anti-β2GPI IgG measured using a BIO-FLASH® assay. ***p<0.0001; **p=0.0013

FIGS. 5A-5F show results demonstrating that COVID-19 immunoglobulins induce proinflammatory and procoagulant genes in monocytes and endothelial cells. FIG. 5A shows induction of mRNA expression in MM1 cells by immunoglobulin (10 μg/ml) isolated from COVID-19 patients or healthy controls. MM1 were stimulated for 3 hours (TNF) or 1 hour (IRF8, GPB6, F3) with or without the complement inhibitor compstatin (2 μg/ml), inhibitory (αEPCR 1496) or non-inhibitory (αEPCR 1489); mean±SD, n=≥3. ****p<0.0001, ***p=0.0002; one-way ANOVA and Tukey's multiple comparisons test. FIG. 5B shows procoagulant activity (PCA) after stimulation of monocytic MM1 cells by immunoglobulin (10 μg/ml), measured by single-stage clotting assay in triplicates; mean±SD, n=10. ****p<0.0001; one-way ANOVA and Tukey's multiple comparisons test. FIG. 5C shows delayed induction of TNF mRNA in monocytic MM1 cells by stimulation with immunoglobulins (10 μg/ml) from COVID-19 patients or an antiphospholipid syndrome (APS) patient with confirmed aPL crossreactive with (32 GPI for 12 hours; mean±SD, n=≥2; ****p<0.0001; one-way ANOVA and Tukey's multiple comparisons test. FIG. 5D shows TNF and F3 mRNA expression in HUVEC stimulated for 3 h with immunoglobulins (10 m/ml); mRNA expression was normalized to the positive control LPS; mean±SD, n≥3, ****p<0.0001; one-way ANOVA and Tukey's multiple comparisons test. FIGS. 5E-5F show inhibition of immunoglobulin induction (10 μg/ml isolated from one representative COVID-19 patient) of TNF (FIG. 5E) and F3 (FIG. 5F) mRNA in HUVEC by compstatin (2 μg/ml), inhibitory (αEPCR 1496) or non-inhibitory (αEPCR 1489), or the endosomal ROS inhibitor niflumic acid (NFA) 10 μg/mL); mRNA expression was normalized to the positive control LPS; mean±SD, n=≥3; ****p≤0.0001, ***p≤0.001. T-test or Mann-Whitney test following Shapiro-Wilk test for normal distribution.

FIGS. 6A-6D show prevention of procoagulant and proinflammatory monocyte activation by aPL with the TF inhibitor rNAPc2. FIG. 6A shows endosomal ROS production by aPL HL5B in the presence or absence of rNAPc2. FIG. 6B shows induction of the indicated mRNAs after 1 hour of stimulation of MM1 monocytic cells with HL5B in the presence or absence of rNAPc2. FIG. 6C shows that rNAPc2 prevents TF and TNFα induction by COVID-19 patient IgG. FIG. 6D shows that rNAPc2 does not influence the induction of the prototypic interferon regulated GBP6 by COVID-19 IgG fractions.

FIG. 7 shows analysis of inferior vena cava thrombosis via intravital imaging, as described in Example 5. Mean+SD,****P<0.0001; ANOVA with Tukey's multiple comparisons test.

### DETAILED DESCRIPTION

The present disclosure is based at least in part on the discovery that NAPc2 influences two detrimental host response pathways in COVID-19 infections: activation of TLR7 by viral RNA and prothrombotic effects and inflammatory signaling of antiphospholipid antibodies implicated in COVID-19. Thus, aspects of the present disclosure are directed to methods for treating a subject for a SARS-CoV-2 infection comprising providing NAPc2 to the subject. Further aspects include methods for treatment of COVID-19 Associated Coagulopathy (CAC) in a subject comprising providing NAPc2 to the subject.

### I. Proteins

As used herein, a “protein” or “polypeptide” refers to a molecule comprising at least three amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., NAPc2). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or more contiguous amino acids or nucleic acids, or any range derivable therein, of SEQ ID NO:2 or SEQ ID NO:3.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 (or any derivable range therein) of SEQ ID NO:2 or SEQ ID NO:3.

In some embodiments, the protein or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 (or any derivable range therein) contiguous amino acids of SEQ ID NO:2 or SEQ ID NO:3.

In some embodiments, the polypeptide or protein may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 (or any derivable range therein) contiguous amino acids of SEQ ID NO:2 and/or SEQ ID NO:3 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NO:2 and SEQ ID NO:3.

In some aspects there is a polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, or 83 of any of SEQ ID NO:2 and/or SEQ ID NO:3 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 (or any derivable range therein) contiguous amino acids or nucleotides of any of SEQ ID NO:2 and SEQ ID NO:3.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

A. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

B. Nematode-Extracted Anticoagulant Proteins and NAPc2

Aspects of the present disclosure are directed to compositions comprising one or more Nematode-extracted Anticoagulant Proteins (NAPs) and methods of use thereof. In some embodiments, disclosed are methods for treatment comprising providing a subject with a pharmaceutical composition comprising one or more NAPs. In some embodiments, NAPs of the present disclosure are one or more of those described in U.S. Pat. No. 5,866,542, incorporated herein by reference in its entirety. In some embodiments, the disclosed methods and compositions comprise NAPc2. In some embodiments, the disclosed methods and compositions comprise NAPc2/proline.

As used herein, NAPc2, (SEQ ID NO:2) describes a single-chain, non-glycosylated 85 amino acid protein (MW=9732 Da). “rNAPc2” describes a recombinant NAPc2 protein. Without wishing to be bound by theory, NAPc2 is understood to inhibit the activity of the TF:Factor (F) VIIa complex that initiates the TF pathway in coagulation, and other key pathways, through the formation of a quaternary complex following binding to zymogen FX. Also disclosed herein are variants of NAPc2. In some embodiments, the disclosed therapeutic compositions comprise a protein having at least or at most 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% sequence identity to NAPc2 (SEQ ID NO: 2), or any range or value derivable therein. In some embodiments, disclosed are compositions comprising NAPc2/proline. “NAPc2/proline” (SEQ ID NO:3) refers to a variant of NAPc2, which has been modified to add a proline residue to the C-terminus of the sequence of NAPc2. In some embodiments, the disclosed therapeutic compositions comprise a protein having at least or at most 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% sequence identity to NAPc2/proline (SEQ ID NO: 3), or any range or value derivable therein.

TABLE 1
NAPc2 and NAPc2 variant sequences

| | SEQ | |
|---|---|---|
| | ID | |
| Protein | NO | Sequence |
| NAPc2 | 2 | KATMQCGENEKYDSCGSKECDKKCKYDGVEEEDDEE |
| | | PNVPCLVRVCHQDCVCEEGFYRNKDDKCVSAEDCEL |
| | | DNMDFIYPGTRN |
| NAPc2/ | 3 | KATMQCGENEKYDSCGSKECDKKCKYDGVEEEDDEE |
| proline | | PNVPCLVRVCHQDCVCEEGFYRNKDDKCVSAEDCEL |
| | | DNMDFIYPGTRNP |

### II. Viruses

A. Coronaviruses

Aspects of the disclosure relate to compositions and methods for treatment of an infection with one or more viruses. In some embodiments, a virus is a DNA virus. In some embodiments, a virus is an RNA virus. In particular embodiments, a virus is from the family Coronaviridae. Alternatively, in some embodiments, a virus is not from the family Coronaviridae. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and approximately 40 species. They are enveloped viruses having a positive-sense single-stranded RNA genome and a nucleocapsid having helical symmetry. The genome size of coronaviruses ranges from about 26-32 kilobases.

There are four main sub-groupings of coronaviruses, known as alpha, beta, gamma, and delta, and seven coronaviruses that can infect people. The four most common coronaviruses utilize humans as their natural host and include: 229E (alpha coronavirus); NL63 (alpha coronavirus); OC43 (beta coronavirus); HKU1 (beta coronavirus). Three other human coronaviruses are: MERS-CoV (the beta coronavirus that causes MERS); SARS-CoV (the beta coronavirus that causes SARS); and SARS-CoV-2 (the novel coronavirus that causes coronavirus disease 2019, or COVID-19).

The interaction of the coronavirus spike protein with its complement host cell receptor is central in determining the tissue tropism, infectivity, and species range of the virus. Coronaviruses mainly target epithelial cell receptors. They can be transmitted by aerosol, fomite, or fecal-oral routes, for example. Human coronaviruses infect the epithelial cells of the respiratory tract, while animal coronaviruses generally infect the epithelial cells of the digestive tract. For example, coronaviruses such as SARS-CoV-2 can infect, via an aerosol route, human epithelial cells of the lungs by binding of the spike protein receptor binding domain (RBD) to an angiotensin-converting enzyme 2 (ACE2) receptor on the cell surface.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and including the species of severe acute respiratory syndrome-related coronavirus. In specific embodiments, the disclosure encompasses treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the species of severe acute respiratory syndrome-related coronavirus, including at least SARS-CoV-2. In specific embodiments, the virus has a genome length between about 29000 to about 30000, between about 29100 and 29900, between about 29200 and 29900, between about 29300 and 29900, between about 29400 and 29900, between about 29500 and 29900, between about 29600 and 29900, between about 29700 and 29900, between about 29800 and 29900, or between about 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC 045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular embodiments, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% sequence identity to any of these viruses. In particular embodiments, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% sequence identity to any of these viruses. As one specific example, the present disclosure includes methods of treatment or prevention of infection of a virus having a genome sequence of SEQ ID NO:1 (represented by GenBank® Accession No. NC 045512; origin Wuhan, China) and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% sequence identity to SEQ ID NO:1.

### III. Treatment of SARS-CoV-2 and Associated Conditions

Aspects of the present disclosure are directed to methods for treatment of a subject having a coronavirus infection, including any coronavirus disclosed herein, for example a SARS-CoV-2 infection. Certain aspects are directed to treatment of conditions associated with a SARS-CoV-2 infection, including thrombosis and coagulopathies, e.g., COVID-19 associated coagulopathy (CAC). Certain aspects of CAC are described in, for example, Iba T, et al., Expert Rev Respir Med. 2021 Mar. 14:1-9 and Memar Montazerin S, et al., Infez Med. 2021 Mar. 1; 29(1):1-9, incorporated herein by reference in their entirety. In some embodiments, disclosed are methods for treatment of a subject having a SARS-CoV-2 infection comprising providing a therapeutically effective amount of NAPc2 or a variant thereof.

As used herein, “coronavirus infection” refers to an infection caused by any Coronaviridae family member. For example, coronavirus infections can include but are not limited to SARS-CoV-2 infections. Thus, aspects of the present disclosure are directed to methods comprising treatment of a subject suffering from, suspected of having, or at risk for developing a coronavirus infection. In some embodiments, the coronavirus infection is a SARS-CoV-2 infection.

Certain embodiments are directed to treatment of subjects having one or more symptoms of a SARS-CoV-2 infection. Symptoms of a SARS-CoV-2 infection include, but are not limited to, fever, dry cough, fatigue, shortness of breath or difficulty breathing, loss of appetite, aches, chills, sore throat, diarrhea, loss of taste, and loss of smell. In some embodiments, a subject has been diagnosed with a SARS-CoV-2 infection. In some embodiments, a subject has not been diagnosed with a SARS-CoV-2 infection. In some embodiments, a subject is at risk for having or developing a SARS-CoV-2 infection.

In some embodiments, the subject was previously treated for a coagulopathy. In some embodiments, a composition comprising NAPc2 is provided to a subject having a SARS-CoV-2 infection, where the subject previously suffered from and was treated for a coagulopathy. In some embodiments, the subject was treated with an anticoagulant. In some embodiments, the anticoagulant was not NAPc2. In some embodiments, the subject was determined to be resistant to the previous treatment for the coagulopathy.

In some embodiments, the subject is suffering from a coagulopathy. The coagulopathy may be CAC. The coagulopathy may not be CAC. In some embodiments, the subject is determined to have a coagulopathy prior to providing a composition comprising NAPc2. In some embodiments, the subject has elevated D-dimer levels relative to a healthy or control subject, thereby indicating the presence of a coagulopathy. In some embodiments, the subject is determined to have D-dimer levels of at least 10000, 11000, 12000, 13000, 14000, 15000, 16000, 17000, 18000, 19000, or 20000 μg/L, or any range or value derivable therein. In some embodiments, the subject does not have elevated D-dimer levels. In some embodiments, the subject was determined to have elevated fibrinogen levels relative to a control or healthy subject, thereby indicating the presence of a coagulopathy. In some embodiments, the subject does not have an elevated fibrinogen level. In some embodiments, the subject was determined to have an elevated interleukin-6 (IL-6) level relative to a control or healthy subject. In some embodiments, the subject does not have an elevated IL-6 level. In some embodiments, a composition comprising NAPc2 is provided to a subject having a SARS-CoV-2 infection and suffering from a coagulopathy. In some embodiments, the subject is not suffering from a coagulopathy.

In some embodiments, the subject is suffering from disseminating intravascular coagulation (DIC). In some embodiments, a composition comprising NAPc2 is provided to a subject having a SARS-CoV-2 infection and suffering from DIC. In some embodiments, the subject is not suffering from DIC.

In some embodiments, the subject is suffering from thrombosis. In some embodiments, a composition comprising NAPc2 is provided to a subject having a SARS-CoV-2 infection and suffering from thrombosis. In some embodiments, the subject is not suffering from thrombosis.

In some embodiments, a subject treated for a SARS-CoV-2 infection and/or associated conditions is at least, is at most, or is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 years of age, or any range derivable therein. In some embodiments, a composition comprising NAPc2 is provided to a subject having a SARS-CoV-2 infection and at least 40, at least 50, at least 60, at least 70, at least 80, or at least 85 years of age. In some embodiments, the subject is at least 65 years of age.

In some embodiments, a subject treated for a SARS-CoV-2 infection and/or associated conditions has one or more risk factors associated with a severe SARS-CoV-2 infection (e.g., an infection resulting in decompensation and/or death). Example risk factors include, but are not limited to, breathing disorders (e.g., asthma, chronic respiratory disease, etc.), diabetes, and cardiovascular disease. In some embodiments, a composition comprising NAPc2 is provided to a subject having a SARS-CoV-2 infection and one or more risk factors associated with a severe SARS-CoV-2 infection.

In some embodiments, a subject is administered a pharmaceutical composition comprising NAPc2 or a variant thereof. The pharmaceutical composition may be administered in a therapeutically effective amount. In some embodiments, the NAPc2 is provided at a dose of at least, at most, or about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, or 15.0 μg/kg or mg/kg, or any range or value derivable therein. The pharmaceutical composition may be administered to a subject every day, every other day, every third day, or every fourth day. In some embodiments, the pharmaceutical composition is administered to the subject on a first day, a third day, and a fifth day. The NAPc2 or variant thereof may be administered at the same dose on each day or at different doses. In some embodiments, the NAPc2 or variant thereof is provided at a first dose on a first day and a second dose on each subsequent day of treatment. In some embodiments, the NAPc2 or variant thereof is provided at a first dose on a first day and a second dose on a third day and a fifth day. In some embodiments, the NAPc2 or variant thereof is provided at a dose of about 7.5 μg/kg on a first day, about 5.0 μg/kg on a third day, and about 5.0 μg/kg on a fifth day.

Aspects of the disclosure are directed to administration of one or more antiviral therapies. Antiviral therapies contemplated herein include any therapy that treats, prevents, and/or improves or alleviates the symptoms of one or more viral infections, including a SARS-CoV-2 infection. In some embodiments, an antiviral therapy of the disclosure is NAPc2 or a variant thereof. In some embodiments, the antiviral therapy is NAPc2. In some embodiments, the antiviral therapy is NAPc2/proline. Additional antiviral therapies are known in the art and contemplated herein, examples of which include remdesivir, COVID-19 convalescent plasma, and anti-SARS-CoV-2 spike protein antibodies (e.g., bamlanivimab).

### IV. Administration of Therapeutic Compositions

The therapy provided herein may comprise administration of a single therapeutic agent (e.g., NAPc2) or a combination of therapeutic agents, such as NAPc2 and an additional anticoagulant. The therapies may be administered in any suitable manner known in the art. For example, each of a first and second therapy may be administered sequentially (at different times) or concurrently (at the same time). In some embodiments, the first and second therapies are administered in a separate composition. In some embodiments, the first and second therapies are in the same composition.

Embodiments of the disclosure relate to compositions and methods comprising therapeutic compositions. A therapeutic composition may comprise a single therapeutic agent (e.g., NAPc2) or multiple different therapeutic agents. The different agents may be administered in one composition or in more than one composition, such as 2 compositions, 3 compositions, or 4 compositions. Various combinations of the agents may be employed.

The therapeutic agents of the disclosure (e.g., NAPc2, NAPc2/proline) may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the therapeutic agent (e.g., NAPc2, NAPc2/proline) is administered subcutaneously. In some embodiments, the therapeutic agent (e.g., NAPc2, NAPc2/proline) is administered intravenously. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 1 μg/kg to 200 μg/kg can affect the protective capability of these agents. It is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. In some embodiments, an effective dose is at least, at most, or about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 μg/kg. Furthermore, such doses can be administered at multiple times during a day, and/or on multiple days, weeks, or months.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### V. General Pharmaceutical Compositions

In some embodiments, pharmaceutical compositions are administered to a subject. Different aspects may involve administering an effective amount of a composition to a subject. In some embodiments, NAPc2 (or NAPc2 proline) may be administered to the subject to protect against or treat a condition (e.g., a SARS-CoV-2 infection, COVID-19 associated coagulopathy). Such compositions may be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, or in addition, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—NAPc2 Upregulates Tissue Factor and TNFα in TLR7-Stimulated Monocytes

RNA viruses, including Ebola, Dengue and SARS-CoV-2, cause severe coagulopathic syndromes and activate the RNA sensing toll like receptor (TLR)⁷. Stimulation of monocytes with the TLR7 agonist R848 induces the coagulation initiator tissue factor (TF) and the proinflammatory cytokine TNFα. The late induction of reactive oxygen species (ROS) and the upregulation of TNF by R848 is specifically blocked with intracellularly acting, small molecule direct FXa inhibitor (Rivaroxaban)¹, but not FXa (NAP5) or thrombin (hirudin) protein inhibitors primarily targeting the extracellular space². Induction of TNFα by R848 requires the TF cytoplasmic domain and protease activated receptor (PAR) 2, a potential drug target in Sar-CoV-2 infection³. Thus, signaling by TF-PAR2 directly supports monocyte responses by TLR7 agonists, raising the question of which specific anticoagulants are beneficial in suppressing adverse effects of viral pathogens.

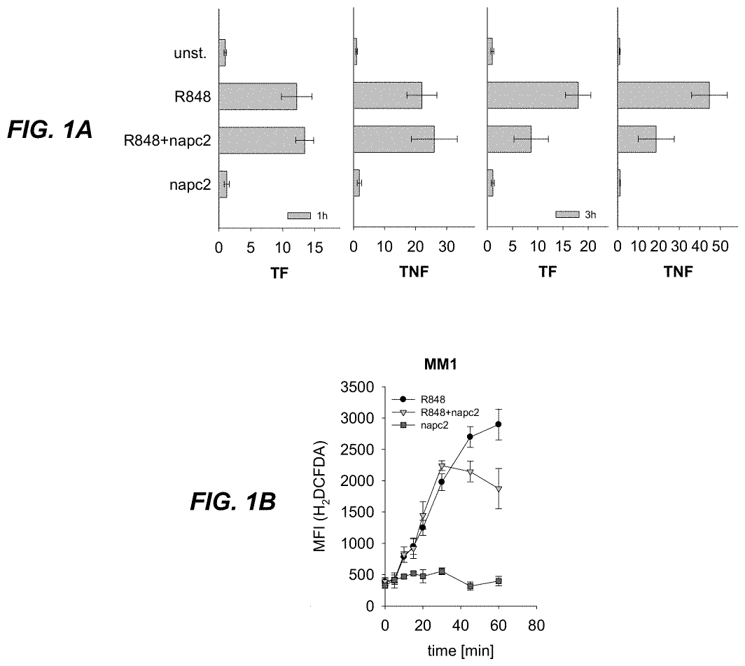

TF inhibition with the hookworm-derived inhibitor NAPc2 in Ebola-infected non-human primates markedly attenuates coagulation activation and inflammation and increases survival⁴ and NAPc2 attenuates inflammation in challenged human volunteers⁵. NAPc2 is similar in its inhibitory mechanism to the physiological TF pathway inhibitor (TFPI), but NAPc2 also recognizes the substrate FX for more rapid and efficient shutdown of TF activity⁶. Given the role of TF-PAR2 in TLR7 signaling, the effect of NAPc2 on the time dependent upregulation of TF and TNF in TLR7-stimulated monocytes was tested. Whereas NAPc2 had no effect on the initial TF and TNFα induction by the TL7 agonist R848, NAPc2 attenuated the sustained induction of procoagulant and proinflammatory responses, as well as the sustained FXa-dependent production of ROS measured by 2′,7′-dichlorodihydrofluorescein diacetate (H₂DCFDA) fluorescence (FIGS. 1A and 1B). Thus, NAPc2 attenuates FXa and PAR2-dependent proinflammatory and procoagulant effects of a TLR7 agonist, providing a mechanism for anti-inflammatory effects.

### Example 2—NAPc2 Prevents TLR7/8-Dependent Antiphospholipid Signaling

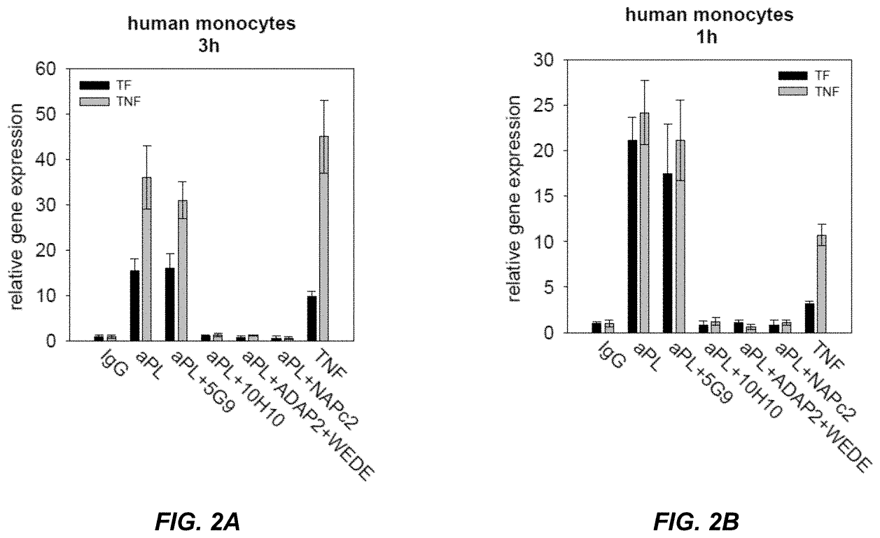

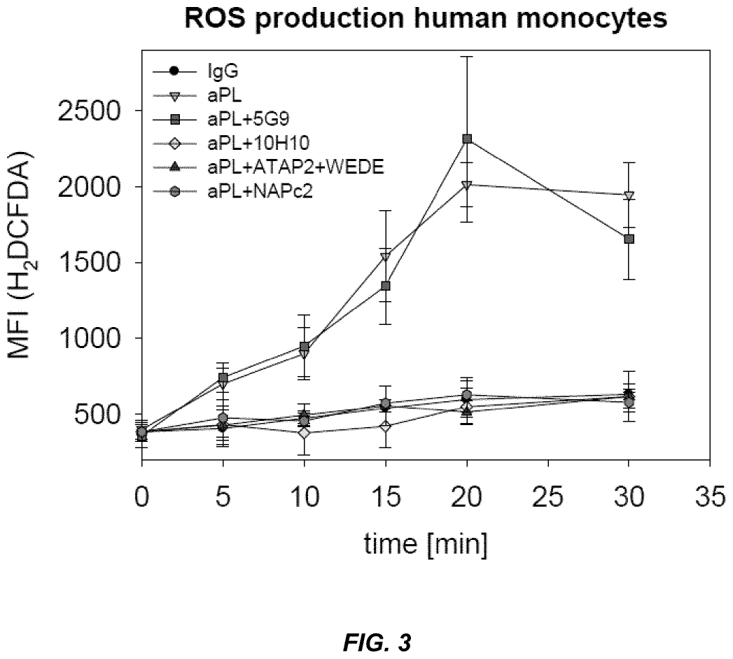

NAPc2 was evaluated for the ability to inhibit the induction of antiphospholipid antibodies in patients with thrombosis during acute infection⁷. Human antiphospholipid antibodies (e.g. HL5B) dissociate a TFPI inhibited TF-FVIIa-FXa complex to initiate proinflammatory cell signaling and the upregulation of TF². Because NAPc2 can restore inefficient TFPI inhibition due to a very similar inhibitory mechanism⁶, the inventors evaluated whether NAPc2 prevented antiphospholipid signaling that is also dependent on TLR7/8⁸. NAPc2 completely blocked the induction of TF, TNFα (FIGS. 2A and 2B) and ROS (FIG. 3) by the antiphospholipid antibody HL5B in human monocytes. These responses were dependent on TF generating thrombin for PAR1 activation which was blocked by antibody to PAR1 (ATAP2/WEDE). As shown in FIGS. 2A, 2B, and 3, NAPc2 was as effective as an antibody to TF (10H₁₀), which blocks TF activation in antiphospholipid-induced thrombosis⁹ and has proven in vivo activity in antiphospholipid syndrome-related pregnancy loss¹⁰. Thus, NAPc2 influences inflammatory signaling of antiphospholipid antibodies implicated in COVID-19.

### Example 3—Evaluation of NAPc2 Efficacy in COVID-19 Patients

Patients are selected for evaluation based on the inclusion and exclusion criteria outlined in Table 2. Patients are randomized to be treated with NAPc2 or heparin. NAPc2-treated patients are given NAPc2 at a dose of 7.5 m/kg subcutaneously (SC) on day 1, and then SC doses of 5 μg/kg on days 3 and 5. Various endpoints are measured as outlined in Table 3.

TABLE 2
Criteria for COVID-19 patient evaluation

| | |
|---|---|
| Inclusion Criteria | COVID-19+; criteria specific for rNAPc2 study - |
| | hospitalized; D-dimer > upper limit of normal (ULN), |
| | age > 18 years. |
| Exclusion Criteria | moribund; high bleeding risk |

TABLE 3
Endpoint measurement

| | |
|---|---|
| Primary | D-dimer change from baseline to day 7 with rNAPc2 |
| efficacy | compared with heparin |
| Primary | International Society on Thrombosis and Haemostasis |
| safety | (ISTH) major bleeding |
| Secondary | D-dimer change from baseline to day 10; IL-6 change |
| measurements | from baseline to day 7 and day 10; time to recovery, |
| | composite of thrombotic events (MI, stroke, acute limb |
| | ischemia including COVID digits, VTE) and all-cause |
| | mortality, and all-cause mortality within 30 days of |
| | randomization |

### Example 4—Analysis of Lipid-Reactive Antibodies in COVID-19 Patients

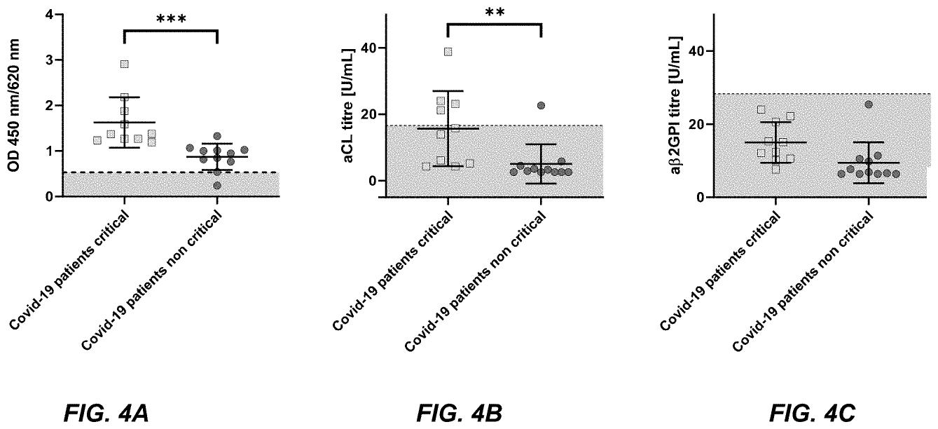

Commercial tests for antiphospholipid antibodies (aPL) are designed to select against lipid-binding aPL associated with infection which might be the reason for the widely discrepant results published on the presence of lipid reactive antibodies in COVID-19 patients until now. The inventors therefore tested serum as well as IgG fractions from hospitalized COVID-19 patients for the presence of lipid-binding aPL using an in house optimized anti-cardiolipin and by QUANTA Flash® automated chemiluminescent immunoassays (Instrumentation Laboratory) for anticardiolipin IgG and anti-β2GPI IgG using the cutoffs determined in a large population-based cohort. In addition, anti-cardiolipin IgG were determined in the in-house ELISA format which does not contain protein cofactors. The cutoff for positivity was determined as the mean plus 3 standard deviations. All but one COVID-19 patients (a non-critical patient) had detectable anti-cardiolipin antibodies in the in-house assay and titers of critical COVID-19 patients were significantly higher than in non-critical cases (FIG. 4A). Less than half of the critical and only 1 of the non-critical cases displayed a positive titer in the commercial anti-cardiolipin assay (FIG. 4B) and all patient's sera had no anti-β2GPI IgG titer (FIG. 4C). None of the patients tested positive for IgM antibodies to cardiolipin or β2GPI in routine clinical laboratory assays.

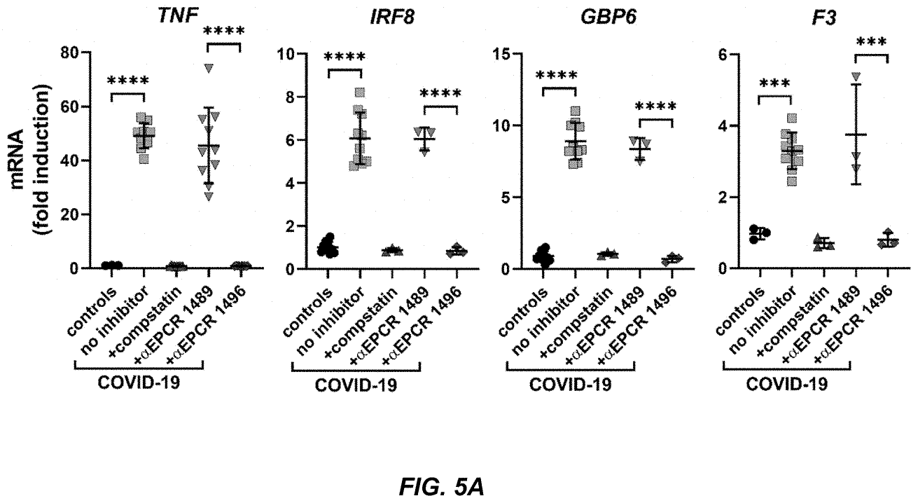

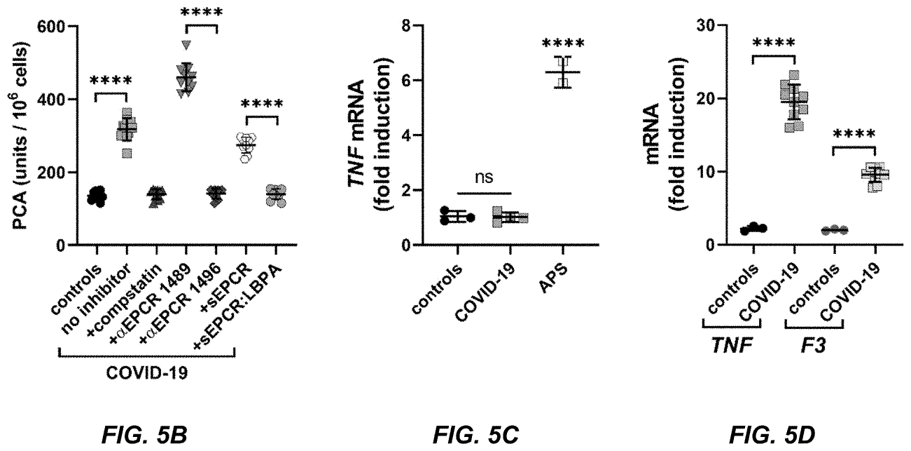

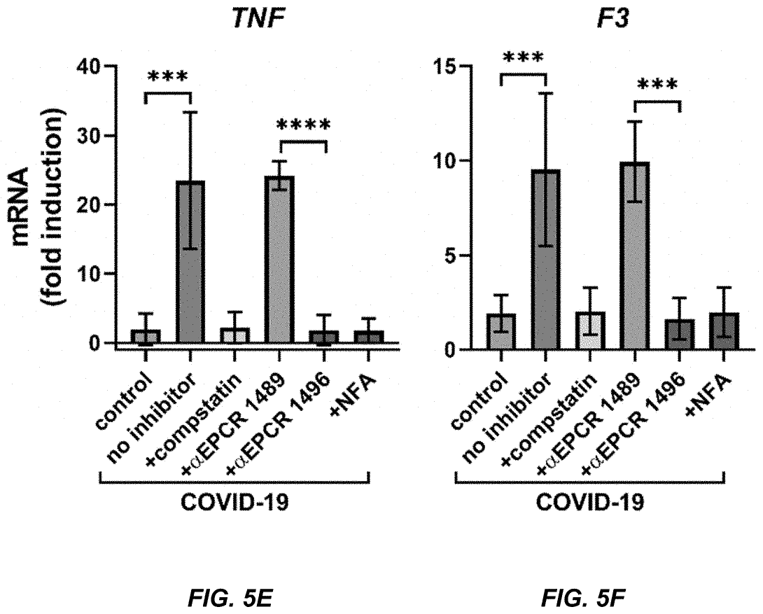

Immunoglobulin isolated from 10 COVID-19 patients induced the expression of TNF, F3, IFR8, and GPB6 in the monocytic cell line MonoMacl (FIG. 5A). All effects were prevented by the complement factor 3 inhibitor compstatin and inhibitory (αEPCR 1496), but not non-inhibitory (αEPCR 1489) monoclonal antibodies against human EPCR.

COVID-19 patient immunoglobulins also rapidly decrypted cell surface TF and this activation was blocked by anti-EPCR and sEPCR loaded with LBPA, but not the unmodified sEPCR carrying the typical structurally bound phosphatidylcholine (FIG. 5B). This indicated that most COVID-19 patients do not develop aPL directed against β2GPI. TNF induction by COVID-19 aPL was no longer observed in monocytes after 12 hours, while β2GPI-reactive IgG from APS patients significantly induced TNF at this time point (FIG. 5C). These data further indicated that no relevant anti-β2GPI reactivity was present in these patient sera.

IgG from COVID-19 patients also rapidly induced TNF and F3 in human umbilical vein endothelial cells (HUVEC) (FIG. 5D). As observed in monocytic cells, this activation was also dependent on complement, EPCR, and endosomal reactive oxygen species (ROS), the latter shown by prevention of endosomal ROS generation by the inhibitor of endosomal superoxide generation niflumic acid (NFA) (FIG. 5E).

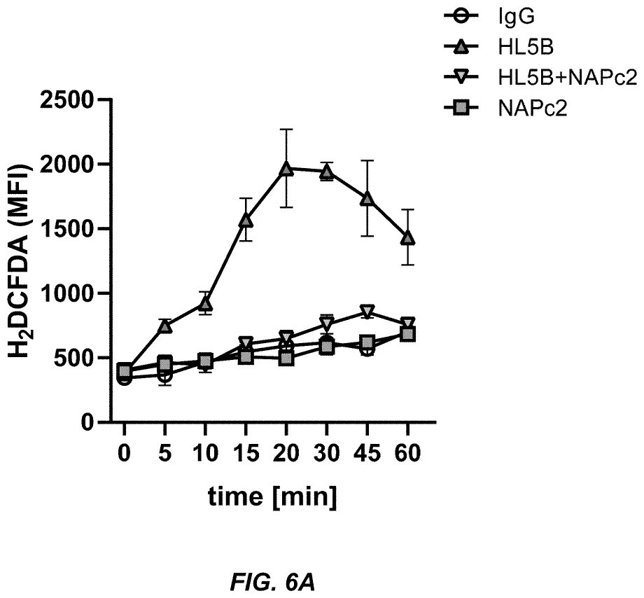

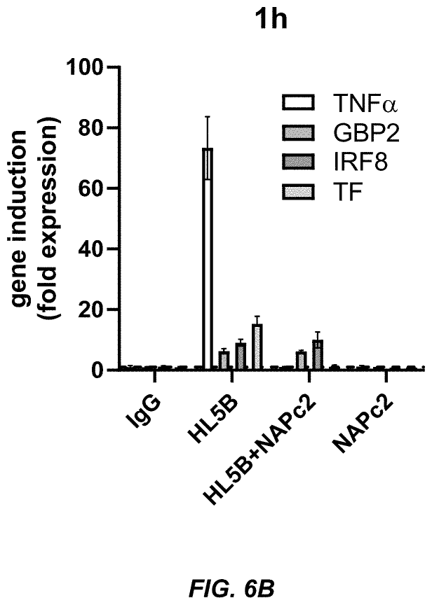

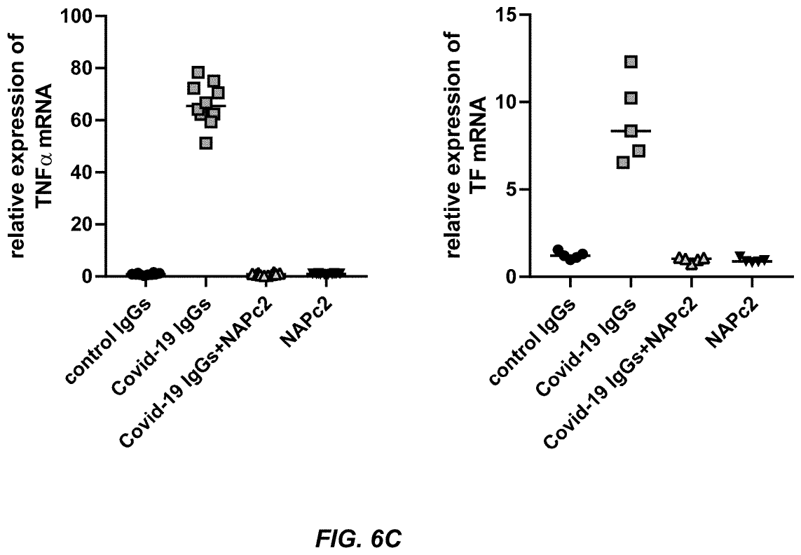

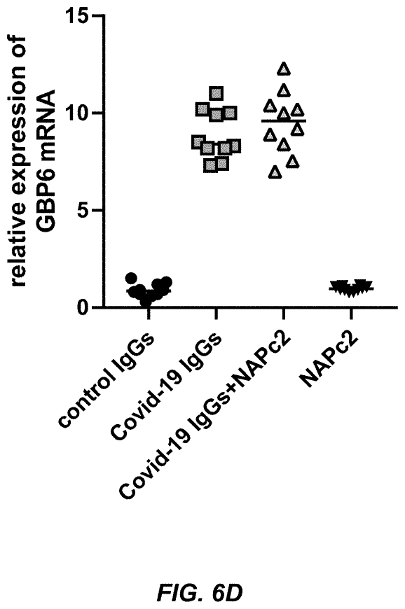

Activation of TF and disruption of an inhibited TF complex is required for aPL endosomal signaling [2]. The inventors therefore evaluated the effects of TF function blockade with the TF-FVIIa-FX inhibitor rNAPc2 on monocyte activation by aPL in the presence of autologous plasma. rNAPc2 blocked aPL HLSB induced endosomal ROS production (FIG. 6A) as well as proinflammatory TNFα and procoagulant TF induction (FIG. 6B) in monocytes. Remarkably, NAPc2 had no effect on the induction of prototypic type I interferon response (IRF8, Gbp2) under the same challenge conditions (FIG. 6B). Similarly, rNAPc2 prevented proinflammatory and procoagulant monocyte TF activation by COVID-19 patient IgG without appreciable effects on the type I interferon response supportive of anti-viral immunity (FIGS. 6C-6D).

### Example 5—Evaluation of the Effect of NAPc2 on Inferior Vena Cava Thrombosis

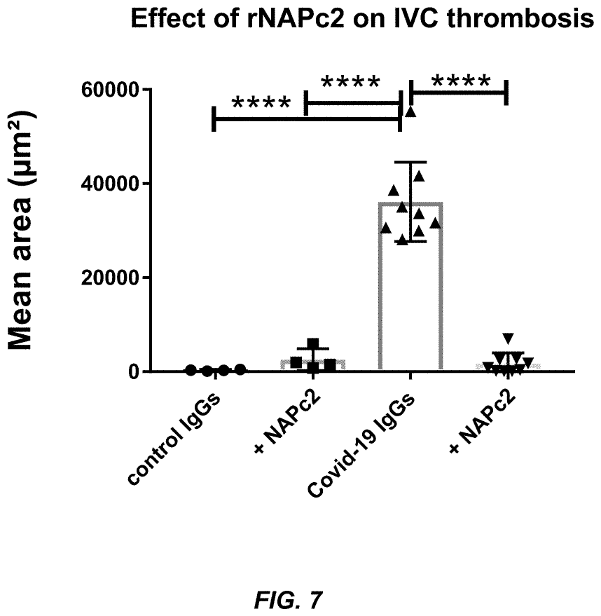

NAPc2 inhibition of COVID-19 IgG amplified inferior vena cava thrombosis was analyzed by applying 1 μg/g body weight rNAPc2 to mice s.c. 30 minutes prior to injecting 10 purified IgG i.v. for intravital imaging, as previously described [2]. Treatment with rNAPc2 significantly reduced COVID-19 IgG-induced inferior vena cava thrombosis compared with the untreated control (FIG. 7).

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Graf, C. et al. Myeloid cell-synthesized coagulation factor X dampens antitumor immunity. Sci Immunol 4, doi:10.1126/sciimmunol.aaw8405 (2019).
2. Muller-Calleja, N. et al. Tissue factor pathway inhibitor primes monocytes for antiphospholipid antibody-induced thrombosis. Blood 134, 1119-1131, doi:10.1182/blood.2019001530 (2019).
3. Gordon, D. E. et al. A SARS-CoV-2 protein interaction map reveals targets for drug repurposing. Nature, doi:10.1038/s41586-020-2286-9 (2020).
4. Geisbert, T. W. et al. Treatment of Ebola virus infection with a recombinant inhibitor of factor VIIa/tissue factor: a study in rhesus monkeys. Lancet 362, 1953-1958 (2003).
5. De Jonge, E. et al. Activation of coagulation by administration of recombinant factor VIIa elicits interleukin 6 (IL-6) and IL-8 release in healthy human subjects. Clin. Diagn. Lab Immunol 10, 495-497 (2003).
6. Bergum, P. W. et al. Role of zymogen and activated factor X as scaffolds for the inhibition of the blood coagulation factor VIIa-tissue factor complex by recombinant nematode anticoagulant protein c2. J. Biol. Chem 276, 10063-10071 (2001).
7. Zhang, Y. et al. Coagulopathy and Antiphospholipid Antibodies in Patients with Covid-19. N Engl J Med 382, e38, doi:10.1056/NEJMc2007575 (2020).
8. Prinz, N. et al. Antiphospholipid antibodies induce translocation of TLR7 and TLR8 to the endosome in human monocytes and plasmacytoid dendritic cells. Blood 118, 2322-2332 (2011).
9. Muller-Calleja, N. et al. Complement C5 but not C3 is expendable for tissue factor activation by cofactor-independent antiphospholipid antibodies. Blood Adv 2, 979-986, doi:10.1182/bloodadvances.2018017095 (2018).
10. Redecha, P., Franzke, C. W., Ruf, W., Mackman, N. & Girardi, G. Activation of neutrophils by the Tissue Factor-Factor VIIa-PAR2 axis mediates fetal death in antiphospholipid syndrome. J Clin Invest 118, 3453-3461 (2008).

1-84. (canceled)

85. A method for treating a subject for a SARS-CoV-2 infection, the method comprising providing to the subject a therapeutically effective amount of a pharmaceutical composition comprising nematode anticoagulant protein c2 (NAPc2) or NAPc2/proline.

86. The method of claim 85, wherein the pharmaceutical composition comprises NAPc2.

87. The method of claim 85, wherein the pharmaceutical composition comprises NAPc2/proline.

88. The method of claim 85, further comprising providing an additional antiviral therapy to the subject.

89. The method of claim 85, wherein the subject was determined to have symptoms of COVID-19.

90. The method of claim 85, wherein the subject does not have symptoms of COVID-19.

91. The method of claim 85, wherein the pharmaceutical composition is provided via subcutaneous injection.

92. The method of claim 85, wherein the pharmaceutical composition is provided via intravenous infusion.

93. The method of claim 85, wherein the pharmaceutical composition is provided via oral administration.

94. The method of claim 85, wherein the pharmaceutical composition is provided to the subject every other day.

95. The method of claim 85, wherein the NAPc2 or NAPc2/proline is provided at a dose of between about 5 μg/kg and about 10 μg/kg.

96. The method of claim 95, wherein the NAPc2 or NAPc2/proline is provided at a dose of about 7.5 μg/kg.

97. The method of claim 95, wherein the NAPc2 or NAPc2/proline is provided at a dose of about 5 μg/kg.

98. The method of claim 85, wherein the method comprises providing NAPc2 or NAPc2/proline at a dose of about 7.5 μg/kg on a first day, providing NAPc2 or NAPc2/proline at a dose of about 5 μg/kg on a third day, and providing NAPc2 or NAPc2/proline at a dose of about 5 μg/kg on a fifth day.

99. The method of claim 85, wherein the subject is suffering from a coagulopathy.

100. The method of claim 85, wherein the subject was determined to have an elevated D-dimer level relative to a control or healthy subject.

101. The method of claim 85, wherein the subject is suffering from thrombosis.

102. A method for treating a subject for COVID-19 associated coagulopathy (CAC), the method comprising providing to the subject a therapeutically effective amount of a pharmaceutical composition comprising NAPc2 or NAPc2/proline.

103. The method of claim 102, wherein the pharmaceutical composition is provided via subcutaneous injection.

104. The method of claim 102, wherein the pharmaceutical composition is provided via intravenous infusion.

105. The method of claim 102, wherein the pharmaceutical composition is provided via oral administration.

106. The method of claim 102, wherein the pharmaceutical composition is provided to the subject every other day.

107. The method of claim 102, wherein the NAPc2 or NAPc2/proline is provided at a dose of between about 5 μg/kg and about 10 μg/kg.

108. The method of claim 107, wherein the NAPc2 or NAPc2/proline is provided at a dose of about 7.5 μg/kg.

109. The method of claim 102, wherein the subject was determined to have elevated an D-dimer level relative to a control or healthy subject.

110. The method of claim 102, wherein the subject is suffering from thrombosis.

111. A method for treating a subject for a SARS-CoV-2 infection, the method comprising providing to the subject a pharmaceutical composition comprising NAPc2/proline at a dose of 7.5 μg/kg.

112. The method of claim 111, further comprising, at least 24 hours after providing the pharmaceutical composition to the subject, providing to the subject an additional pharmaceutical composition comprising NAPc2/proline at a dose of 5 μg/kg.

113. The method of claim 111, wherein the subject was determined to have an elevated D-dimer level relative to a control or healthy subject.

114. A method for treating a subject for a SARS-CoV-2 infection, the method comprising:
(a) subcutaneously providing a first composition comprising NAPc2/proline at a dose of 7.5 μg/kg on a first day;
(b) subcutaneously providing a second composition comprising NAPc2/proline at a dose of about 5 μg/kg on a third day; and
(c) subcutaneously providing a third composition comprising NAPc2/proline at a dose of about 5 μg/kg on a fifth day,
wherein the subject was determined to have an elevated D-dimer level relative to a control or healthy subject.


SEQUENCE LISTING (3 sequences)

SEQ ID NO: 1 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - protein, 84 aa, Ancylostoma caninum
KATMQCGENEKYDSCGSKECDKKCKYDGVEEEDDEEPNVPCLVRVCHQDCVCEEGFYRNKDDKCVSAEDCELDNMDFIYPGTRN

SEQ ID NO: 3 - protein, 85 aa, Ancylostoma caninum
KATMQCGENEKYDSCGSKECDKKCKYDGVEEEDDEEPNVPCLVRVCHQDCVCEEGFYRNKDDKCVSAEDCELDNMDFIYPGTRNP